(19) United States
(12) Patent Application Publication
(10) Pub. No.: US 2004/0147723 A1
(43) Pub. Date: Jul. 29, 2004

(54) Moringa seed proteins

(76) Inventors: Nicolas Mermod, Buchillon (CH); Ian William Marison, Echandens (CH); Stephen Christos Dorries, Lausanne (CH); Evelyne Meyer, Lausanne (CH); Mougli Suarez, Ecublens (CH)

Correspondence Address: NIXON & VANDERHYE, PC, 1100 N GLEBE ROAD, 8TH FLOOR, ARLINGTON, VA, 22201-4714, US

(21) Appl. No.: 10/483,835

(22) PCT Filed: Jul. 19, 2002
(86) PCT No.: PCT/CH02/00401

(30) Foreign Application Priority Data
Jul. 19, 2001 (WO) PCT/CH01/00451
Feb. 15, 2002 (WO) PCT/CH02/00092

(51) Int. Cl. C07K 14/415

(52) U.S. Cl. 530/370; 435/69.1; 435/252.33; 435/320.1

(57) ABSTRACT

The invention concerns a new family of proteins obtained from Moringa seeds or derived from Moringa seed proteins. Those proteins can be used for different purposes such as coagulation agents for the water treatment and/or as antibiotic agents. This new protein family consists of at least 5 sub-families: A first being obtained according to a recombinant process. All other sub-families being obtained according to specific extraction processes. Proteins obtained according to the extraction process in the present text.

### FIELD OF THE INVENTION

The present invention relates to proteins which are obtained from Moringa seeds or derived from Moringa seed proteins.

More precisely the invention concerns a family of proteins obtained from Moringa seeds or derived from Moringa seed proteins which may be used for different purposes such as coagulation agents for water treatment.

### STATE OF THE ART

Moringa genus comprises some 14 plant species, in particular Moringa oleifera.

Moringa seeds are primarily used to obtain an edible oil which may be extracted using a mechanical press.

It has been found that the seeds of Moringa contain water soluble, low molecular weight, highly basic proteins that can act as flocculating agents in contaminated water treatment. Some parts of these active compounds have been isolated and identified (Gassenschmidt, U., Jany, K.-D., Tauscher, B., and Niebergall, H. (1995). Isolation and characterization of a flocculating protein from Moringa oleifera Lam. Biochim. Biophys. Acta 1243, 477-481). One protein moiety, MO2.1, has been determined and it was shown that it contains 60 amino acids with a high content in glutamine, arginine and proline.

International patent application WO 99/48512 (LABORATOIRES SEROBIOLOGIQUES) discloses the use in the cosmetic or in the dermatological field of at least one protein moiety, e.g. MO2.1, extracted from Moringa seeds.

International patent application WO 00/46243 (OPTIMA ENVIRONNEMENT SA) relates to proteins and to a specific process for preparing these proteins which are extracted from Moringa seeds and which can act as coagulation agents.

### SUMMARY OF THE INVENTION

The invention concerns a new family of proteins obtained from Moringa seeds or derived from Moringa seed proteins. These proteins can be used for different purposes such as coagulation agents for the water treatment and/or as antibiotic agents, in particular they efficiently kill human pathogens, including antibiotic-resistant clinical isolates.

This new protein family consists of at least 5 sub-families:

A first being obtained according to a recombinant process. All other sub-families being obtained according to specific extraction processes. Proteins obtained according to the extraction process of the invention will be referred as E proteins in the present text.

In the present text, the term antibiotic means in particular bacteriostatic, bactericidal, antifungal or toxic to any other type of cell, and antiviral.

It has to be mentioned that previous attempts to express a recombinant form of Moringa proteins is already disclosed in the prior art (Tauscher, B. (1994). Water treatment by flocculant compounds of higher plants. Plant Res. and Dev. 40, 56-70) and to demonstrate an associated coagulation activity were not met with success.

The inventors of the present invention have developed a process to obtain an active bacterially-produced recombinant protein.

E Proteins have different structures than the ones of Moringa proteins disclosed in the prior art.

Proteins according to the invention can act as coagulation agents not only in water but also in other fluids such as blood, milk or any other edible liquid. They can also be used in the pharmaceutical and in the cosmetic field, in particular in all indications cited in WO 99/48512.

Some examples related to the present invention will be discussed hereafter together with the following figures:

### BRIEF DESCRIPTION OF THE FIGURES

FIG. 1. Schematic representation of Flo expression and purification.

FIG. 2. Flo protein expression.

FIG. 3. Assay for the coagulation activity of Flo.

FIG. 4. Effect of Flo on E.coli culture growth.

FIG. 5. shows SDS-PAGE (polyacrylamide gel electrophoresis) of extracts of seed proteins, oil body proteins and synthetic peptides from Moringa oleifera.

FIG. 6. shows SDS-PAGE (polyacrylamide gel electrophoresis) of extracts of seed proteins, and synthetic peptides from Moringa oleifera extracted under reducing conditions.

FIG. 7. population analysis profile in 50 mM pH 7 KPO4.

FIG. 8. population analysis profile in MHB nutrient broth.

FIG. 9. killing of S. aureus P8 by Flo against in MHB nutrient broth and pH 7.50 mM KPO4 buffer.

FIG. 10. DNA sequence and corresponding peptide sequence of H1 H2 and H3.

### DETAILED DESCRIPTION OF THE INVENTION

In the following examples the invention will be detailed with a recombinant process and with a specific E proteins extraction process from Moringa seeds. The resulting proteins will be compared with PHYTOFLOC which is a commercial preparation of Moringa seed extracts. Briefly, for obtaining PHYTOFLOC a ground presscake of Moringa seeds is mixed with saltwater at 1:5 w/v ratio. The extract is filtered and heated at 75° C. Precipitated solids are removed by centrifugation and the clarified liquor is concentrated by filtration through 5 kD cut-off membranes.

Materials and Methods

Plasmids

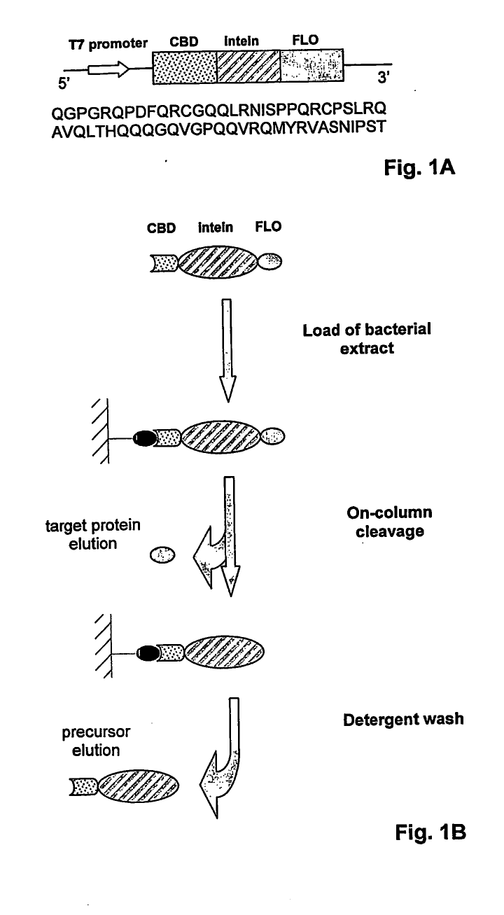

A DNA sequence was designed to encode the MO2.1 polypeptide sequence (Gassenschmidt et al., 1995, see FIG. 1A). The recombinant form of this polypeptide is termed Flo in the present text. The double strand oligonucleotide was synthesized using a PCR assembly strategy, as described by Horton et al. (1989). The oligonucleotide sequence was designed so that its codons are optimized for E.coli expression and so that SapI and PstI restriction sites are located at its extremities. The pTYB11 plasmid of the IMPACT expression system (Intein Mediated Purification with an Affinity Chitin-binding Tag system, New England Biolabs, Inc.) was selected for cloning and expressing the Moringa seed Flo protein in E.coli. The oligonucleotide was ligated to SapI/PstI digested pTYB11 vector so that the sequences encoding the N-terminus of the target protein Flo, an internal protein self-cleavage site (intein), and chitin binding domain, are fused. Positive clones were verified by sequencing.

Protein Expression and Purification.

The pTYB vectors use a Lac repressor-controlled T7 promoter and the laci gene to provide stringent control of the fusion gene expression. Binding of the lac repressor to the lac operator sequence located Immediately downstream of the T7 promoter suppresses basal expression of the fusion gene in the absence of IPTG induction. The E.coli was ER2566 as it carries a chromosomal copy of the T7 RNA polymerase gene under control of the lac promoter. To induce expression of the fusion protein, 0.3 mM IPTG was added to an exponentially growing culture at an A₆₀₀ of 0.5-0.6 during 2 hours at 27° C., with agitation at 200 rpm. The bacterial culture, extract preparation and purification conditions as well as the used buffers were as recommended by the manufacturer (New England Biolab). In brief, 1.5 liter bacteria culture volume (A₆₀₀=0.5-0.6) was centrifuged and cells were lysed by sonication. Extracts were clarified by centrifugation and loaded onto an equilibrated chitin beads (50-100 μm particle size) column. After washing, the column was filled with 50 mM DTT containing buffer which was incubated in the column during 40 hours at room temperature, to allow for self-cleavage of the Intein-containing fusion peptide. Flo was eluted and its presence confirmed by gel electrophoresis. Finally, precursor protein was eluted with stripping buffer and the column recycled.

Total cell protein extracts were analyzed using 10% SDS-Page gels (Laemmli, 1970). For protein quantification, gels were stained using cypro-orange and analyzed using scanning software (STORM 840, Pharmacia Amersham biotech.). This allowed the ratio of fusion protein to total extract to be estimated by direct comparison with various quantities of BSA loaded in parallel. Due to its small size, the eluted Flo polypeptide was analyzed through the tricine-sodium dodecyl sulfate-polyacrylamide gel electrophoresis (Schagger et al, 1987). For gel fixing and staining, a protocol suitable for small basic proteins was followed (Steck et al., 1980)

Chemically Synthesized Proteins

Some recombinant proteins of the invention and E proteins were synthesized according to standard procedures.

### E PROTEINS—EXAMPLE 1

Preparation of a Crude Oil Body Protein Extract from Moringa oleifera Seeds:

Dry seeds of Moringa were dehusked manually and homogenized using a Polytron for 40 seconds at maximum power in 4 volumes of cold (4° C.) homogenization buffer (0.15M Tricine buffer pH 7.5 containing 1 mM EDTA, 10 mM KCl, 1 mM MgCl₂, 2 mM dithiothreitol and 0.6M sucrose). The homogenate was filtered through a nylon membrane (20 μm pore size) to remove large particles and seed debris. Clarified homogenate was diluted with 1 volume flotation buffer (0.15 M Tricine pH 7.5 containing 0.4 M sucrose, 1 mM EDTA, 10 mM KCl, 1 mM MgCl₂ and 2 mM dithiothreitol) and centrifuged for 30 minutes at 10,000 g. Oil bodies were collected from the surface of the centrifuged suspension and added to 0.5 volumes of the homogenization buffer containing 2M NaCl to re-suspend. A further 0.5 volumes of homogenization buffer, containing 2 M NaCl and 0.25 M sucrose in place of 0.6 M sucrose, were added to the surface of the oil body suspension followed by centrifugation for 30 minutes at 10,000 g. Oil bodies were collected from the surface of the centrifuged suspension and re-suspended in 0.5 volumes of homogenization buffer followed by re-centrifugation for 30 minutes at 10,000 g. The washing procedure was repeated and the oil bodies re-suspended in homogenization buffer to give a final concentration of 100 mg per liter (in general achieved by addition of 20 volumes of homogenization buffer to oil bodies and stored at 4° C.

The crude oil body protein extracts prepared in this way have been analyzed by SDS gel electrophoresis after the addition of SDS.

### E PROTEINS—EXAMPLE 2

Preparation of a Purified Oil Body Protein Extract from Moringa oleifera Seeds:

Crude oil body proteins prepared according to Example 1 were purified by recovery of the oil bodies from the surface of the buffer after the final centrifugation step followed by the addition of an organic solvent such as acetone, hexane or other to remove the associated triacylglycerides. Solvent-treated oil body proteins were then recovered by centrifugation for 2 minutes at 13,500 g. Oil body proteins were recovered from the surface of the centrifuged samples, washed with organic solvent (acetone, hexane or other) and re-centrifuged under the same conditions. A second washing step was then carried out by resuspending the oil body proteins in diethyl ether and re-centrifuged for 2 minutes at 13,500 g. Oil body proteins were recovered form the last centrifugation step and resuspended in ultra-high purity (UHP) water containing 1.5 volumes of a 2:1 mixture of chloroform in methanol. The latter was centrifuged for 4 minutes at 10,000 g and the purified oil body proteins isolated from the water solvent interface. The isolated proteins were then washed twice with the water/chloroform/methanol solution, centrifuged for 4 minutes at 10,000 g. The purified oil body proteins were then recovered from the water-solvent interface and a dried protein preparation made by evaporation of the organic solvent under an atmosphere of nitrogen gas. The purified oil body proteins prepared in this way could be stored at 4° C. indefinitely.

The purified oil body protein extracts prepared in this way have been analyzed by SDS gel electrophoresis after the addition of SDS.

### E PROTEINS—EXAMPLE 3

Preparation of a Crude Seed Protein Extract from Moringa oleifera Seeds:

Dry seeds of Moringa were dehusked manually and homogenized using a Polytron for 40 seconds at maximum power in 4 volumes of cold (4° C.) homogenization buffer (0.15M Tricine buffer pH 7.5 containing 1 mM EDTA, 10 mM KCl, 1 mM MgCl₂ and 0.6M sucrose). The homogenate was filtered through a nylon membrane (20 μm pore size) to remove triglycerides and oil bodies. The remaining solids material was collected and termed presscake. Seed proteins were extracted by re-suspending the presscake in 5 volumes of salt solution followed by stirring for 1 hour. Extracted seed proteins were recovered by centrifugation for 5 minutes at 1,500 g followed by decantation through a fine cotton cloth. Decanted seed protein extracts were heated to 85° C. with gently stirring and subsequently cooled to room temperature before centrifugation for 5 minutes at 1,500 g. The supernatant was collected and could be stored at room temperature.

The crude seed protein extracts prepared in this way have been analyzed by SDS gel electrophoresis after the addition of SDS.

### E PROTEINS—EXAMPLE 4

Preparation of a Purified Seed Protein Extract from Moringa oleifera Seeds:

The procedure is followed according to Example 3 except that a reducing agent, such as 1% dithiothreitol (DTT) was added to the extraction salt solution. In this way the disulphide bonds responsible for the conformation of many multimeric and monomeric proteins were reduced and were removed during subsequent centrifugation and filtration steps.

The seed protein extracts prepared in this way have been analyzed by SDS gel electrophoresis after the addition of SDS.

E Protein Sequence

Sequencing one of the E proteins showed that one of its terminal ends starts with the sequence RGPAFRR.

Coagulation Test

The test was carried out in a 2 ml volume in a spectrophotometer cell (104QS/HELLMA). To evaluate the coagulation activity, 100 mg/ml suspension of 3.5-7 μm diameter glass beads (Sheriglass 5000, Potters-Ballotini) were diluted in 50 mM phosphate buffer, pH=7.0, to mimic turbid water. Stirring was kept continuously at 800 rpm and OD 500 nm was measured each second (LabVIEW sfotware/National Instruments Corporation) in a Perkin-Elmer 552 spectrophotometer. After 5 minutes of continuous stirring the compound to be tested was added to a final concentration of 20 μg/ml, and stirring was continued for 15 minutes. Active compounds were diluted before the test either in phosphate buffer 10 mM, pH=7.0 (E proteins, PHYTOFLOC and bacterially produced Flo) or in distilled water (synthetic Flo).

Analytical Methods

To quantify the flocculation efficiency a linear regression was performed on time points corresponding to four minutes before the addition of the flocculating preparation (basal sedimentation) and four minutes after the addition of the flocculating preparation (coagulation-mediated sedimentation). The difference between the coagulation-mediated and basal sedimentation was calculated by subtraction in OD per minutes and multiplied by 1000 (Δ slope).

Antibiotic Effect

E.coli ER2566 was grown in LB medium to the exponential phase (A₆₀₀=0.5-0.6) at 37° C. as above. The culture was centrifuged and resuspended in a same volume of 10 mM phosphate buffer pH=7.0, and E proteins, PHYTOFLOC (2 mg/ml), synthetic Flo (0.1 mg/ml to 2 mg/ml) carrier (buffer) or BSA (2 mg/ml) were added. After 2 hours incubation at 37° C., LB was added to the bacteria culture to obtain A₆₀₀=0.1. All cultures were incubated at 37° C. at 200 rpm and the culture growth was followed through A₆₀₀ measurements.

Many other micro-organisms were tested, comprising Staphylococcus aureus, Streptococcus pyrogenes, Enterococcus faecalis, Bacillus subtilis, Klebsiella oxytoca, Pseudomonas aeruginosa and in a second group of tests also Legionella pneumophilia, Mycobacterium abscessus/chelonae and Mycobacterium fortuitum.

Structure of Flo

Further the structure of Flo has been analyzed.

Results

Preliminary Remark.

Previous work on the coagulating activity associated with other Moringa seed extracts indicated that the activity copurifies with small molecular weight proteins. The sequence of one of them was determined as positively charged 6 kDa polypeptide (Tauscher, 1994). However, previous attempts to express a recombinant form of this protein and to demonstrate an associated coagulation activity were not met with success.

Protein Cloning, Expression and Purification.

Using the protein sequence, we reconstructed a synthetic gene that would be optimal for expression in E.coli of the recombinant Moringa seed protein, which we termed Flo. Given the highly positively charged nature of Flo, expression as a fusion protein was chosen. The expression vector was designed so that the Flo protein is expressed as a fusion with an heterologous polypeptide consisting of an intein sequence and a chitin binding domain (FIG. 1A). The chitin binding domain allows for easy separation of the fusion protein from the rest of the bacterial proteins, using chitin-containing chromatography resins. Inteins are amino acid sequences that allow post-translationally cleavage of precursor proteins, in a controlled autocatalytic process, when thiol containing compounds are added (see Perler, 2000 for a review, FIG. 1B).

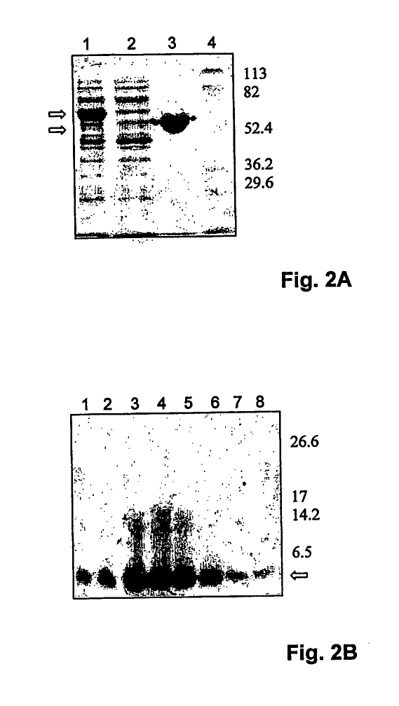

The pTYB vectors of the IMPACT expression system uses a lac repressor controlled T7 promoter-driven system to achieve high levels of expression and tight transcriptional control in E.coli. Upon addition of lac repressor inhibitors, the lac repressor system is derepresed allowing the expression of the T7 RNA polymerase and liberating the lac operator sequence downstream of the T7 promoter. Over-express of a fusion protein of the expected size was specifically obtained from extracts of bacteria frown under inducing conditions (FIG. 2A, lane 1, and data not shown). Quantification of the total and specific protein content indicated that approximately 30% of the protein content of induced cells consist of the Flo fusion protein. This preparation was loaded onto a chitin beads-containing column. Contaminating bacterial proteins were washed away and the fusion protein was cleaved by incubation with thiol-containing reducing compound. This allowed the elution and recovery of native bacterially expressed Flo polypeptide (FIG. 2B), freed from the chitin binding portion of the fusion protein that remained associated with the chromatography resin. Finally, the precursor protein, comprising the intein sequence and chitin binding domain was eluted (FIG. 2A, lane 3). The bacterially produced Flo polypeptide was quantified directly on gel by direct comparison with known amounts of a chemically synthesized Flo polypeptide. Approximately 1 mg of purified Flo protein was obtained per liter of bacterial culture.

E Proteins

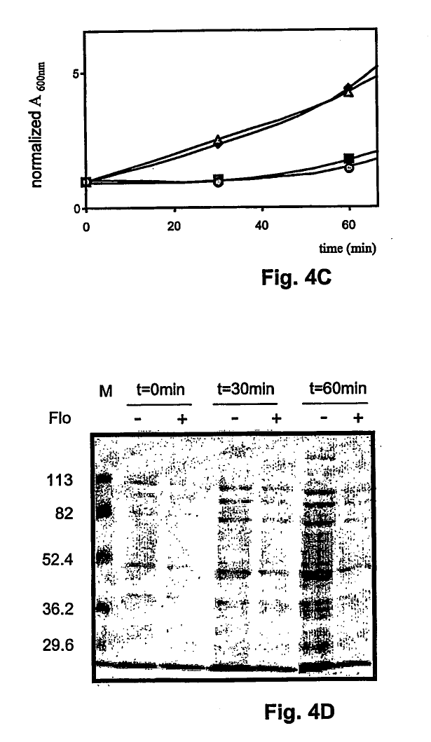

FIG. 5 shows SDS-PAGE (polyacrylamide gel electrophoresis) of extracts of seed proteins, oil body proteins and synthetic peptides from Moringa oleifera.

Lane 1: Standard proteins (Sigma);

Lane 2: Seed proteins extracted under reducing conditions;

Lane 3: Total oil body proteins extracted under reducing conditions (undiluted);

Lane 4: Total oil body proteins extracted under reducing conditions (10-fold dilution);

Lane 5: Total oil body proteins extracted under reducing conditions (100-fold dilution);

Lane 6: Empty;

Lane 7: Seed proteins extracted under non-reducing conditions;

Lane 8: Total oil body proteins extracted under non-reducing conditions (undiluted);

Lane 9: Total oil body proteins extracted under non-reducing conditions (10-fold dilution);

Lane 10: Total oil body proteins extracted under non-reducing conditions (100-fold dilution).

Gel indicates that seed protein extracts and oil body protein extracts from Moringa oleifera contain similar proteins. The proteins extracted under non-reducing conditions contain one major protein fraction with a molecular weight of approximately 17 kDaltons whereas proteins extracted under reducing conditions contain two major protein fractions with molecular weights of approximately 6.5 and 5.5 kDaltons.

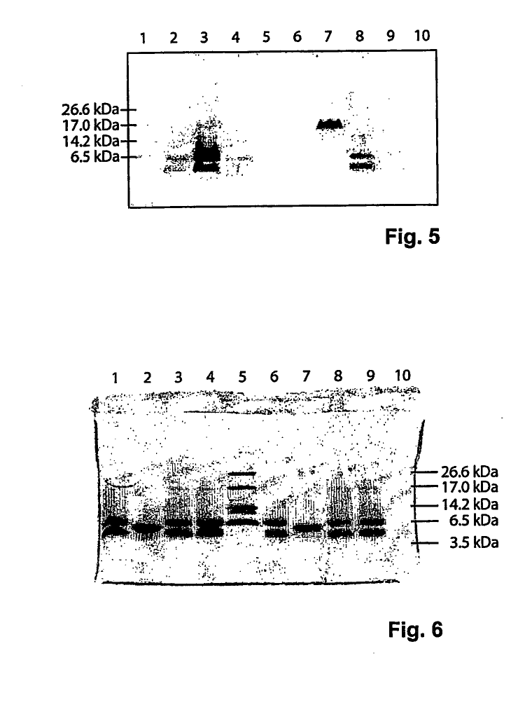

FIG. 6 shows SDS-PAGE (polyacrylamide gel electrophoresis) of extracts of seed proteins, and synthetic peptides from Moringa oleifera extracted under reducing conditions.

Lane 1: Seed protein extracts from de-fatted seeds (presscake);

Lane 2: Synthetic peptide (sequence according to Gassenschmidt et al., 1995);

Lane 3: Seed protein extracts from whole ground seeds;

Lane 4: Seed protein extracts from de-fatted seeds (presscake) after dialyzes against water;

Lane 5: Ultra low molecular weight protein standards (Sigma);

Lane 6: Seed protein extracts from de-fatted seeds (presscake);

Lane 7: synthetic peptide (sequence according to Gassenschmidt et al., 1995);

Lane 8: Seed protein extracts from whole ground seeds;

Lane 9: Seed protein extracts from de-fatted seeds (presscake) after dialysis against water. All extracts loaded onto gel at 2.5 μg total protein.

Results show that the synthetic peptide produced with the reported sequence of a protein extracted from Moringa oleifera (Gassenschmidt et al., 1995) migrates on the SDS-PAGE at a position corresponding to a molecular weight (Lanes 2 and 7) of approximately 6.0 kDaltons and does not correspond to either of the fractions obtained by the extraction procedure covered by the present patent application. All protein fractions, from all extracts exhibited flocculation activity.

Coagulation Activity

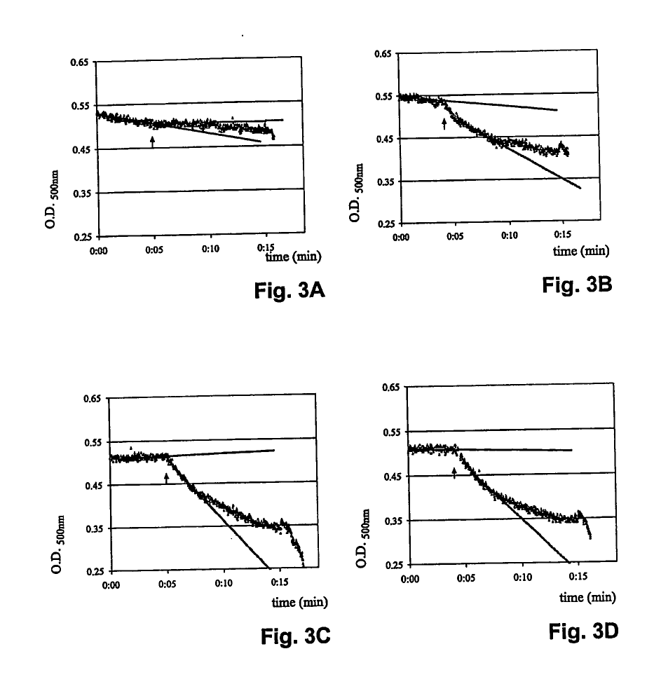

To assess particle coagulation properties of Flo, a suspension of glass beads under continuous mixing was used to mimic turbid water. Sedimentation was estimated by following the decrease in optical density resulting from the scattering of light by the beads in suspension. After 5 minutes of recording of the basal speed of particle sedimentation, the compound to be tested was added and the variation in slope was calculated as follows: Δslope=(slope value after adding. flocculant—slope value before adding flocculant)×1000 . Little sedimentation occurred before or after the addition of buffer (FIG. 3A). However, efficient coagulation was noted when using PHYTOFLOC (FIG. 3B). Similar sedimentation rates was observed when using either similar amounts of the chemically synthesized Flo (FIG. 3C), bacterially produced Flo (FIG. 3D) or E proteins.

Interestingly, under these test conditions, the specific coagulation activity of the synthetic, recombinant Flo or E proteins were higher than that estimated using PHYTOFLOC. As the latter contains several major polypeptides, it is possible that the component directly responsible for the coagulation effect is under-represented as compared to other protein preparations. An alternative explanation is that the seed extract may contain inhibitors of coagulation. Indeed, direct size comparison of recombinant or synthetic Flo with PHYTOFLOC indicated that it does not match the polypeptides detected in PHYTOFLOC, and that Flo is likely to consist of a fragment of a naturally occurring polypeptide. In any case, these results indicate that Flo is highly active in the coagulation assay.

Antibiotic Effect

Moringa seed extracts were shown previously to flocculate bacteria and to possess antimicrobial activity (Eilert et al., 1981; Madsen et al, 1987). The active principle of the flocculation activity was not identified, while the antimicrobial activity was ascribed to plant-synthesized derivatives of benzyl isothiocyanates, a known antibacterial compound. Nevertheless, we set up to characterize potential effects of the Flo polypeptide and of E proteins on E.coli. To do so, bacteria from exponentially growing cultures were incubated with the peptides of the invention. Visual inspection revealed that the peptide did aggregate the bacteria, as indicated by the appearance of defined partides or flocs, which size grew over time. In the absence of agitation, the bacteria incubated with the peptide quickly sedimented, unlike bacteria incubated with buffer only which remained in suspension. The spreading of the cultures incubated with peptides of the invention on solid growth media yielded less viable colonies when compared to control cultures. These results indicate that peptides of the invention can flocculate these bacteria Just as they coagulate glass beads.

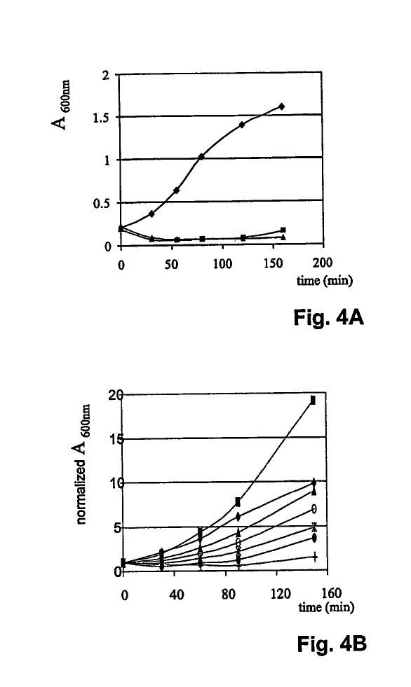

To determine if Flo might have an effect the growth or viability of E.coli, bacterial cells incubated with the peptide were placed in culture medium and incubated under agitation. FIG. 4A shows the bacterial growth of cultures incubated with or without 2 mg/ml of either PHYTOFLOC or Flo. In presence of any one of the latter components, a strong inhibition of the bacterial culture growth was noted. The potential antimicrobial effect of synthetic Flo was studied in more detail in FIG. 4B, which shows a dose-dependent antibacterial growth response. An inhibitory effect is already detectable when bacteria were incubating at low Flo concentration, with an IC₅₀ of approximately 100 μg/ml. Incubation with a high concentration of bovine serum albumin, used as a negative control, indicated that the antibacterial effect is specific to the Flo protein.

After prolonged incubation of the culture, growth resumed, suggesting either that a minority of bacteria showed resistance to a bactericidal activity of Flo, or that Flo acts as a bacteriostatic, and that some bacteria eventually escaped. To distinguish between these two possibilities, a culture of bacteria that was incubated in two cycles with the peptide and that had escape the growth inhibitory effect were collected. To address whether a resistant state of the bacteria had been reached, these cells were challenged again by the addition of Flo (FIG. 4C). Again, the peptide inhibited cell growth, and the effect was indistinguishable from that observed with cells that had never been selected by incubation with the peptide. This indicates that the building of bacterial resistance to the antibiotic effect of Flo does not occur to a detectable level. Therefore, the cell growth that occurred upon prolonged incubation did not occur from an intrinsic resistance of bacteria to Flo, but most likely resulted from the escape of some bacteria, for instance because degradation of the peptide occurred.

To ascertain that E.coli culture growth is truly inhibited by Flo, and that the observed effects does not simply results from a flocculation effect, cell extracts corresponding to different time points were resolved by SDS-PAGE electrophoresis. A slight decrease of the amount of E.coli proteins was noted on several occasions after addition of the peptide (FIG. 4D and data not shown), which may indicate a bactericidal effect of Flo. However, many cells may survive the treatment, at least in these assay conditions, as indicated by the level of proteins remaining in the bacterial pellet. When further incubated in growth medium, bacteria that had been treated with Flo did not synthesize more proteins, in contrast to control cells where protein content strongly increased, coordinately with the increase in the optical density of the culture (FIGS. 4C and D). This indicates that Flo blocks E.coli metabolism and that it possess a bacteriostatic activity. Collectively, these results demonstrate an antibiotic effect of Flo.

Our results show that high yield of the Flo protein can be obtained from E.coli as a fusion protein.

The coagulation test results showed a very efficient coagulation activity of the synthetic and bacterially produced Flo polypeptide, even more than what was obtained using PHYTOFLOC. This effect was observed using two models for water clarification, the coagulation of glass beads and the flocculation of E.coli bacteria. These finding indicates that the Flo peptide, either synthetic or recombinant, possess hallmarks characteristics of efficient water purification.

Inspection of the sequence of the Flo polypeptide indicated that it is very significantly positively charged. This was reminiscent of the so-called peptide antibiotics, which are positively charged peptides found in animal and plants that display a bacteriostatic or bactericidal activity (Schroeder, J.-M. (1999). Epithelial peptide antibiotics. Biochem. Pharmacol. 57, 121-134.). This, taken with the previous demonstration of an antimicrobial activity of Moringa seed extract, prompted us to test a possible antibacterial activity of Flo. We found that the synthetic Flo polypeptide not only flocculates bacteria but that It also prevents bacterial culture growth. This implies that Flo exerts either a bacteriostatic or a bactericidal activity.

As already mentioned, other micro-organisms were tested, this further study is presented here in detail. The purpose of the study was to determine the antibacterial effect of both PHYTOFLOC and Flo against a panel of representative Gram-positive and Gram-negative bacteria. Most antimicrobial assays measure the ability of a given drug to prevent bacterial growth to turbidity in liquid media. By convention, the lowest drug concentration inhibiting growth to “visible” turbidity is referred to as the minimal inhibitory concentration, or MIC (National Committee for Clinical Laboratory Standards, 2000). It provides information on the aptitude of the drug to bloc bacterial division. However, both PHYTOFLOC and Flo share the additional ability to precipitate soluble macromolecules and maybe microorganisms. This may provoke the formation of visible aggregates, resulting in medium turbidity even in the absence of bacterial growth. Therefore, testing the antimicrobial effect of PHYTOFLOC and Flo requires alternative strategies.

A second option consists in exposing the bacteria to the test drug in liquid medium, and then sub-culturing them on nutrient agar plates. The numbers of organisms giving rise to colonies (colony forming units or CFU) represent the surviving organisms and can be compared to the original number of bacteria inoculated into the tubes. Typically, series of tubes containing nutrient broth and 2-fold serial dilutions of the test drug are inoculated with bacteria (final concentration of 10⁵-10⁶ CFU/ml), incubated for 24 h, and then plated to determine the number of surviving bacteria as described. Bacteria in control drug-free medium will have grown by 3-4 log10 CFU/ml in this period of time. In tubes containing inhibitory concentrations of the drug, bacteria are expected to display either no growth, or some decrease in viable counts. In tubes containing bactericidal concentrations of drug, bacteria are expected to have lost ≧3 log10 CFU in viable counts compared to the original inoculum. The lowest drug concentration inflicting such a bactericidal effect is called the minimal bactericidal concentration (MBC) (National Committee for Clinical Laboratory Standards, 2000).

While determining MICs and MBCs by sub-culturing bacteria is an appropriate option, two additional pitfalls must be considered with PHYTOFLOC and Flo. First, it is possible that the compounds aggregate bacteria, thus resulting in falsely low colony counts on the plates. Indeed, while a single bacterial body will give rise to one colony on agar plates, an aggregate of 10 or 100 bacteria will also give rise to one single colony. This may lead to an overestimation of the antibacterial effect (few colonies in spite of large numbers of viable bacteria), but can be reasonably well monitored by light microscopy.

Second, the precipitation of macromolecules from the medium could result in nutrient restriction for the bacteria. Hence, an antibacterial effect could be falsely attributed to the test compound, when it is in fact an indirect effect of energy shortage. This is an unlikely possibility in the present experiments, because laboratory growth media provide carbohydrate and NH₄⁺ in the form of small soluble molecules (e.g., glucose and amino acids) that are apparently not aggregated by the test compounds. As a control, the intrinsic effect of the PHYTOFLOC and Flo was also tested in nutrient-free buffers supplemented or not with the compounds.

Microorganisms, growth conditions and chemicals: The test bacteria are summarized in Table 1. They include several representative Gram-positive and Gram-negative pathogens. The organisms were grown at 37° C. without aeration either in Mueller Hinton broth (MHB; Difco Laboratories, Detroit, Mich.), or on Columbia agar plates (Becton Dickinson Microbiology Systems, Cockeysville, Md.) supplemented with 4% of blood. In certain experiments, tryptic soy agar (TSA; Difco) and brain heart infusion (BHI; Difco) were used to study a possible medium effect. Bacterial stocks were kept frozen at −70° C. in medium supplemented with 10% (vol/vol) of glycerol.

TABLE 1
Bacterial strains used in the study

| Microorganisms | Source |
|---|---|
| Gram-positive | |
| Staphylococcus aureus P8 (methicillin | (Entenza et al., 2001) |
| resistant) | |
| Streptococcus pyogenes ATCC 19615 | NCCLS strain collection |
| Enterococcus faecalis | Clinical isolate (CHUV) |
| Bacillus subtilis | PHYTOFLOC contaminant |
| Gram-negative | |
| Escherichia coli ATCC 25922 | NCCLS strain collection |
| Klebsiella oxytoca | Clinical isolate (CHUV) |
| Pseudomonas aeruginosa | Clinical isolate (CHUV) |

PHYTOFLOC was provided in a stock solution containing 300 mg/ml of protein extract. One stock was kept at 4° C., as recommended by the manufacturer. A second stock was distributed in aliquots that were stored at −20° C. Frozen stocks were thawed prior to utilization and used only once. They were stable with regard to the PHYTOFLOC antibacterial activity. Flo was provided as a dried powder. It was kept at 4° C. and diluted in sterile H₂O immediately prior to use. All other chemicals were reagent grade commercially available products.

Antibacterial susceptibility tests: two-fold serial dilutions of PHYTOFLOC or Flo were distributed in polystyrene tubes containing appropriate buffer or nutrient medium (1 ml for PHYTOFLOC and 0.2 ml for Flo). One experiment was also performed in polypropylene tubes. The tubes were inoculated with a final concentration of ca. 5×10⁵ CFU/ml of the test bacteria and incubated at 37° C. After 24 h of incubation 0.01 and 0.1 ml volumes of each tubes were spread onto nutrient agar as described, and the plates were incubated for an additional 24 h at 37° C. before colony counts. The MIC was defined as the lowest concentration of PHYTOFLOC or Flo inhibiting bacterial growth as compared to the original inoculum. The MBC was defined as the lowest drug concentration resulting in ≧99.9% decrease in viable counts as compared to the original inoculum. Bacteria from tubes containing no drugs and from tubes around the MIC were examined by phase contrast microscopy for bacterial aggregation and gross morphological alterations.

Time-kill experiments: the dynamic of bacterial killing by Flo was studied against one representative Staphylococcus aureus and one Escherichia coli (Table 1) by a described method (Entenza et al., 1997). In brief, bacteria form overnight cultures were inoculated into 10 ml glass tubes containing prewarmed fresh medium to a final concentration of 10⁶ CFU/ml. Immediately after inoculation, Flo was added at concentrations of 2 and 20 mg/ml, respectively. This corresponded to the MIC (2 mg/ml) and 4× the MBC (20 mg/ml) for S. aureus, and to a sub-MIC (2 mg/ml) and 2× the MIC (20 mg/ml) for E coli. At various times before and after drug addition samples of the cultures were removed, serially diluted, and plated on nutrient agar for colony count, as above.

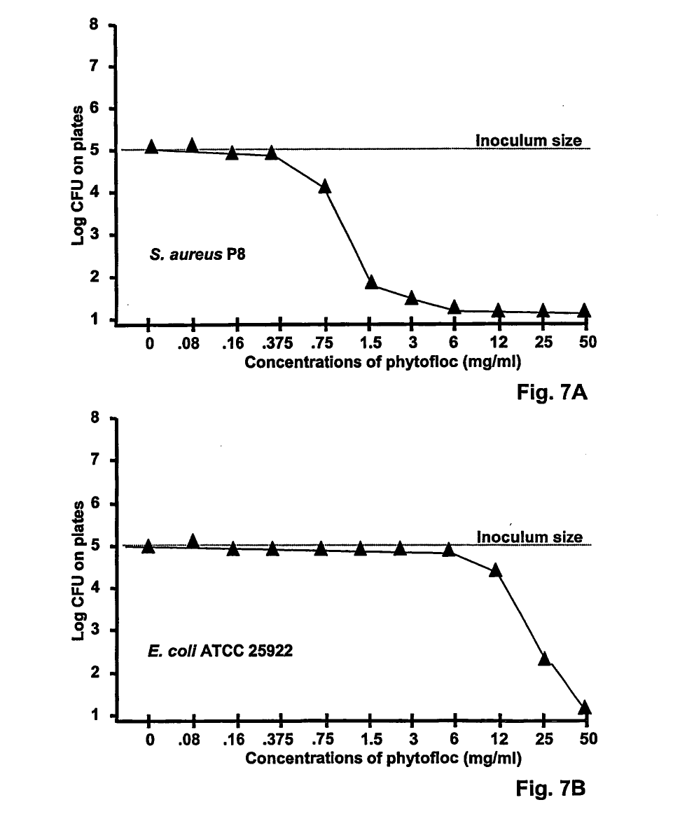

Effect of PHYTOFLOC on bacterial growth and bacterial survival in buffer to determined whether the effect of PHYTOFLOC depended on the presence of nutrient in the solution, S. aureus P8 and E. coli ATCC 25922 were exposed to increasing concentrations of PHYTOFLOC diluted in 50 mM KPO₄ at either pH 6, 7, or 8. Sub-cultures from the tubes were performed after 24 h as described, and the numbers of surviving colonies were determined. FIG. 7 depicts the results obtained at pH 7. Because they were suspended in plain buffer, bacteria did not grow in the control tube without PHYTOFLOC. In the presence of PHYTOFLOC the microorganisms survived up to a concentration of 0.75 mg/l for S. aureus and 50 mg/ml for E. coli, and were killed (loss of ≧3 log10 CFU/ml) at higher concentrations. PHYTOFLOC was slightly less active at pH 6 (curve moved one dilution to the right in FIG. 7) and slightly more active at pH 8 (curve moved one dilution to the left in FIG. 7). Importantly, examination of the bacteria by phase contrast microscopy indicated that the decrease in bacterial viability was not due to aggregation (bacterial clusters were Identical in treated and no-treated tubes), but rather correlated with discrete morphological alterations in the form of bacterial swelling observable in S. aureus.

Thus, PHYTOFLOC appeared genuinely bactericidal in buffer, ruling out a nutrient-dependent artifice. Moreover, there was no obvious medium effect when TSB or BHI were used against S. aureus and E. coli in PHYTOFLOC susceptibility tests.

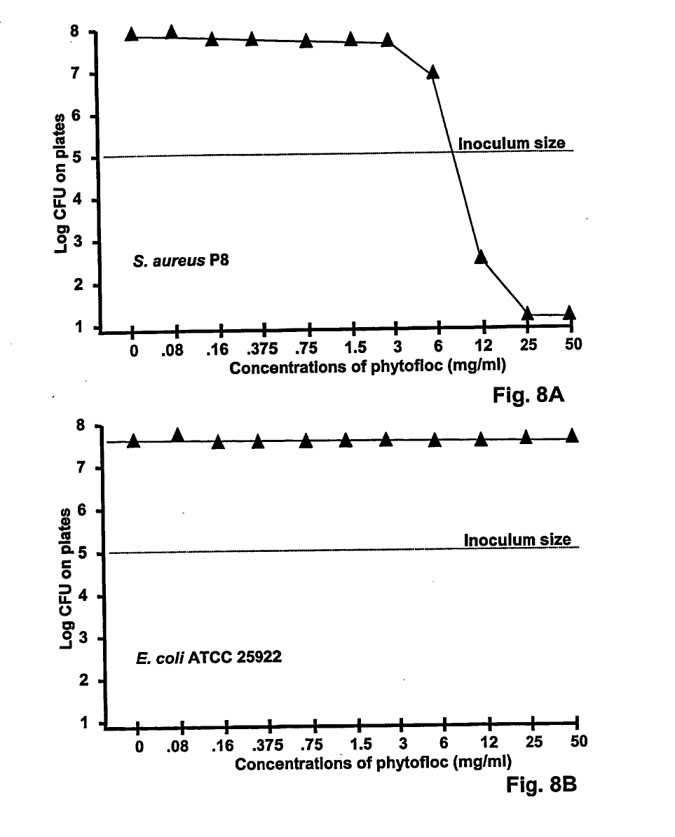

Effect of PHYTOFLOC on bacterial growth and bacterial survival in nutrient broth: FIG. 8 depicts the results of a similar experiment performed in MHB nutrient broth instead of KPO₄ buffer. It can be seen that bacteria grew in most of the tubes, and that larger concentrations of PHYTOFLOC were necessary to achieve inhibition and killing. S. aureus was both inhibited and killed by 12 mg/ml of PHYTOFLOC. In contrast, E. coli was not inhibited by concentration as high as 100 mg/ml. Since this suggested a possible susceptibility difference between Gram-positive and Gram-negative bacteria additional organisms were tested.

Antibacterial activity of PHYTOFLOC and Flo against various bacteria: table 2 presents the MICs and MBCs of the two test compounds for a number of Gram-positive and Gram-negative organisms. The antibacterial activity of PHYTOFLOC was reproducibly observed against both S. aureus and Streptococcus pyogenes. On the other hand, PHYTOFLOC was inactive (at the concentrations tested) against Enterococcus faecalis, Bacillus subtilis, and a panel of Gram-negative bacteria.

Most interestingly, Flo was up to 10-fold more potent than PHYTOFLOC against both S. aureus and S. pyogenes, and successfully inhibited E. coli at 10 mg/l. This indicates that Flo or potential derivatives might overcome the spectrum restriction observed in crude PHYTOFLOC.

TABLE 2
Minimal inhibitory concentrations (MIC) and minimal
bactericidal concentrations of PHYTOFLOC and Flo
against the test organisms

| | PHYTOFLOC | | | Flo | | |
|---|---|---|---|---|---|---|
| | # | | | # | | |
| Bacteria | Tests | MIC* | MBC* | Tests | MIC* | MBC* |
| Gram-positive | | | | | | |
| Staphylococcus aureus | 5 | 9-18 | 12-32 | 4 | 2-5 | 5-10 |
| Streptococcus pyogenes | 2 | 12 | 12-24 | 3 | 2-5 | 2-5 |
| Enterococcus faecalis | 2 | >50 | >50 | ND | ND | ND |
| Bacillus subtilis | 3 | >50 | >50 | ND | ND | ND |
| Gram-negative | | | | | | |
| Escherichia coli | 5 | >50 | >50 | 3 | 10 | >10 |
| Klebsiella oxytoca | 1 | >50 | >50 | ND | ND | ND |
| Pseudomonas aeruginosa | 1 | >50 | >50 | ND | ND | ND |

*given in mg/ml
ND = not determined

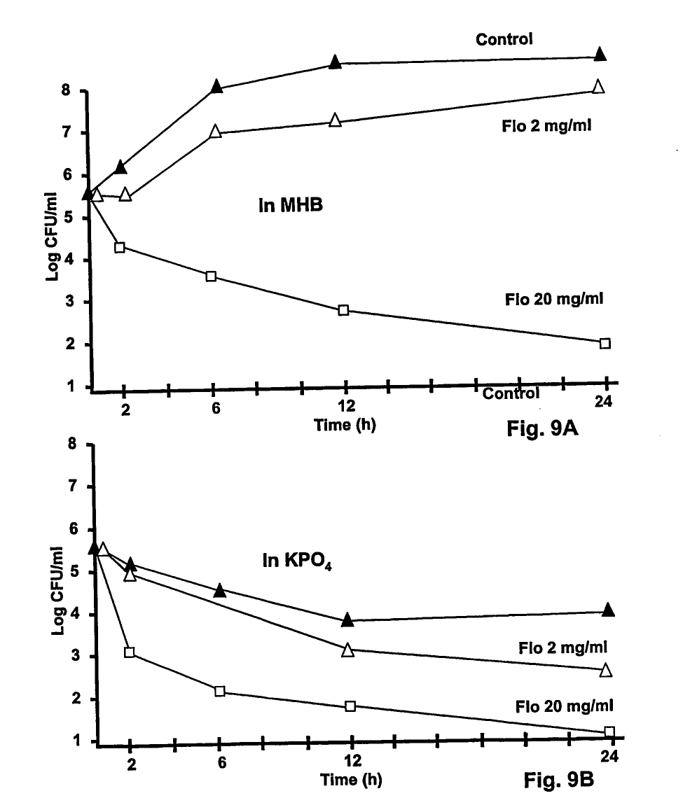

Time-kill experiments: FIG. 9 presents the dynamic of killing during exposure of S. aureus P8 to 2 and 20 mg/ml of Flo in either 50 mM of KPO4 at pH 7, or MHB. At 2 mg/ml, Flo was barely inhibitory. At 20 mg/ml, on the other hand, Flo was clearly bactericidal in both experimental conditions. The same concentrations used against E. coli were not effective in this particular test (data not presented).

In conclusion the experiments presented above clearly identify an antibacterial activity of both PHYTOFLOC and Flo. Although the antibacterial spectrum was restricted, a bacteriostatic and bactericidal effect was reproducibly observed against two major pathogens, i.e., S. aureus and S. pyogenes.

Strain specificity may be useful to treat defined conditions while preserving the normal bacterial flora and avoiding selection of multiple bacterial resistances among commensal organisms. For instance, the t-RNA synthetase inhibitor mupirocin is primarily active against a restricted number of Gram-positive pathogens (including staphylococci and S. pyogenes) and has become a major drug for the eradication of problematic multiresistant staphylococci from chronic carriers, as well as a major drug in superficial skin infection. A similar example can be found with the protein inhibitor fusidic acid, which is almost exclusively aimed at staphylococcal infections. Such compounds are invaluable to decrease the transmission of multidrug resistant organisms including methicillin-resistant as well as the emerging glycopeptide-resistant staphylococci (please note that the S. aureus P8 tested herein is methicillin-resistant).

Two additional aspects of PHYTOFLOC and Flo need to be underlined. One is their bactericidal effect observed against both actively growing bacteria (in nutrient broth) and non-growing bacteria (in KPO₄ buffer). Bacterial killing is a critical property of antimicrobial agents in anatomical sites with restricted immune defenses (a typical situation in skin and mucosal colonization). Yet, very few drugs are able to kill slow-growing or non-growing bacteria, a metabolic state that prevails in most in vivo situations. Most existing antibacterials cannot eradicate the microorganisms by themselves in such situations. Therefore, the unique bactericidal effect of Flo in such condition is remarkable.

The second is the improved activity of Flo over that of crude PHYTOFLOC against both S. aureus and E. coli. indeed, further refining the peptide might allow an improved activity against many more bacteria than the one studied in these first screening tests. A salient example of this is provided by the beta-lactam development. Penicillin G is very active against Gram-positive organisms but not against E. coli. Yet, the mere addition of a single NH₂ group gives rise to ampicillin, which is makes the compound very effective against a number of Gram-negative bacteria.

In conclusion, PHYTOFLOC and its derived cationic Flo share the ability to inhibit and kill S. aureus and S. pyogenes, but appeared less active against gram-negative bacteria. This species restriction may be related to the mode of action of the experimental compounds. From the biomedical point of view the spectrum restriction does not preclude clinical usefulness (e.g., mupirocin against multiresistant staphylococci). Moreover, PHYTOFLOC and Flo demonstrated a unique bactericidal activity against non-growing organisms, which is a potential very important property.

Tests have also been extended to Gram-negative Legionella and further to. Mycobacteria.

The sensibility of Legionella and Mycobacteria to Flo was experienced in the following way:

Source of bacteria: The bacteria were isolated from drinking water of hospitals in Ticino, patient strains were obtained from the laboratory of microbiology at the CHUV.

MIC (minimal inhibitory concentration): MIC was measured with the help of a micro plaque with 96 wells, each containing 100 μl. The growth media were BYEα for L. pneumophilia and TSB for Mycobacterium and were containing a certain concentration of the peptide or antibiotic.

The media were then subjected to twofold dilutions (1 μl of bacterial suspension at a concentration of 5*10⁸ CFU/ml diluted to 5*10⁶ CFU/ml).

The L. pneumophilia culture was incubated at 35° C. and the results were read after 48 and 96 hours. The Mycobacterium culture was incubated at 30° C. for up to seven days.

MIC is the first well with growth.

MBC (minimal bactericidal concentration): 50 μl of the above suspensions were plated on solid media BCYEα or agar with blood. The L. pneumophilia culture was incubated at 35° C. for 48 hours and and up to seven days for Mycobacterium.

The MBC value is the first plate without growth.

The results are shown in table 3 and 4. Table 3 shows the results for L. pneumophilia and table 4 for Mycobacterium abscessus/chelonae and fortuitum.

TABLE 3
results for L. pneumophilia

| | FLO | FLO | | | | |
|---|---|---|---|---|---|---|
| | mg/ml | mg/ml | PHYTOFLOC | PHYTOFLOC | Ciprofloxacin | Ciprofloxacin |
| | MIC | MBC | mg/ml MIC | mg/ml MBC | mg/ml MIC | mg/ml MBC |
| L. | 0.8 | 1.6-3.1 | 4.7-9.4 | 4.7 | 0.03 | 0.03-0.06 |
| pneumophilia | | | | | | |
| serogroup-1 | | | | | | |
| L. | 1.6 | 1.6-3.1 | 9.4 | 9.4 | 0.016 | 0.016-0.03 |
| pneumophilia | | | | | | |
| serogroup 10 | | | | | | |

TABLE 4
results for Mycobacterium abscessus/chelonae and fortuitum

| | N° of days | | |
|---|---|---|---|
| Strain n° | in culture | FLO mg/ml MIC | FLO mg/ml MBC |
| Mycobacterium | | | |
| abscessus/chelonae | | | |
| 30 | 4 | 12.5 | 12.5 |
| 84 | 3 | 12.5 | 12.5 |
| Mycobacterium | | | |
| fortiutum | | | |
| 12 | 3 | 12.5 | 12.5 |
| 48 | 5 | 12.5 | 25 |

It can be concluded that L. pneumophilia is sensitive to Flo and PHYTOFLOC, they show inhibition and bactericidal activity at relatively low concentrations.

The results of table 4 indicate an inhibitory and bactericidal activity of FLO on Mycobacterias at 12.5 mg/ml.

Altogether, our results imply that use of a peptide according to the invention (Flo, E protein) for water clarification as well as for water disinfection is feasible. This indicates that this approach may be a valuable alternative to commonly used.

The MBC value is the first plate without growth.

The results are shown in table 3 and 4. Table 3 shows the results for L. pneumophilia and table 4 for Mycobacterium abscessus/chelonae and fortuitum.

TABLE 3
results for L. pneumophilia

| | FLO | FLO | | | | |
|---|---|---|---|---|---|---|
| | mg/ml | mg/ml | PHYTOFLOC | PHYTOFLOC | Ciprofloxacin | Ciprofloxacin |
| | MIC | MBC | mg/ml MIC | mg/ml MBC | mg/ml MIC | mg/ml MBC |
| L. | 0.8 | 1.6-3.1 | 4.7-9.4 | 4.7 | 0.03 | 0.03-0.06 |
| pneumophilia | | | | | | |
| serogroup 1 | | | | | | |
| L. | 1.6 | 1.6-3.1 | 9.4 | 9.4 | 0.016 | 0.016-0.03 |
| pneumophilia | | | | | | |
| serogroup 10 | | | | | | |

TABLE 4
results for Mycobacterium abscessus/chelonae and fortuitum

| | N° of days | | |
|---|---|---|---|
| Strain n° | in culture | FLO mg/ml MIC | FLO mg/ml MBC |
| Mycobacterium | | | |
| abscessus/chelonae | | | |
| 30 | 4 | 12.5 | 12.5 |
| 84 | 3 | 12.5 | 12.5 |
| Mycobacterium | | | |
| fortiutum | | | |
| 12 | 3 | 12.5 | 12.5 |
| 48 | 5 | 12.5 | 25 |

It can be concluded that L. pneumophilia is sensitive to Flo and PHYTOFLOC, they show inhibition and bactericidal activity at relatively low concentrations.

The results of table 4 indicate an inhibitory and bactericidal activity of FLO on Mycobacterias at 12.5 mg/ml.

Altogether, our results imply that use of a peptide according to the invention (Flo, E protein) for water clarification as well as for water disinfection is feasible. This indicates that this approach may be a valuable alternative to commonly used chemicals. In particular, peptides of the invention are unlikely to have the potential toxic effects associated with chemical water treatment, and Moringa seeds are currently used not only for the traditional treatment of waste water but also for the preparation of various food. Another advantage for water treatment with polypeptides is their good biodegradability, unlike aluminum salts for example, which remain as contaminants of treated waters and of the sedimented materials. Finally, doses around 100 μg/ml of peptides according to the invention act as antibiotic agents, at similar concentration range used for common antibiotics such as β-lactams and others.

Structure of Flo

Bioinformatic approaches predicted the presence of putative alpha-helix structures, the circular dichroism spectroscopy indicated mainly a coiled secondary structure. The sequences respectively called H1, H2 and H3 represent the three domains deducted from the primary structure of Flo.

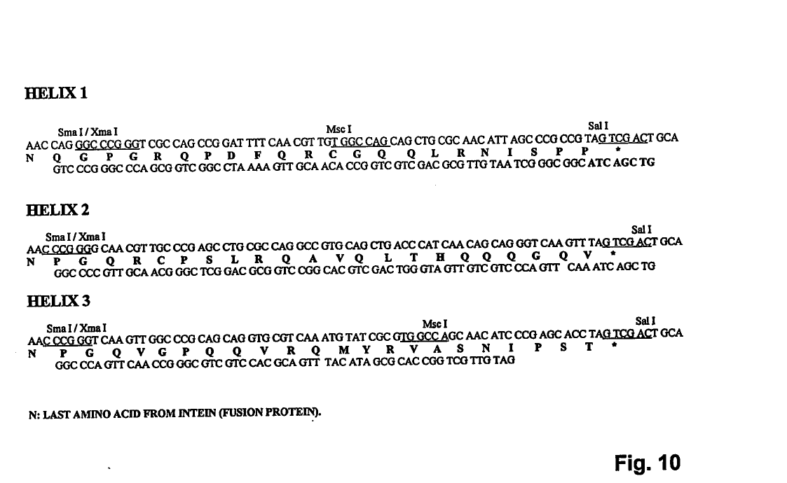

FIG. 10 shows the DNA and corresponding peptide sequences of H1, H2 and H3.

Figures

FIG. 1. Schematic Representation of Flo Expression and Purification.

A. Structure of the Flo fusion protein expression vector. The Flo coding sequence (shaded box) was inserted downstream of sequences encoding the self-cleavage intein protein domain (striped box) fused to the chitin binding domain (CBD, doted box), under the control of a regulated T7 phage promoter. Sequence of the Flo polypeptide, as released from the intein sequence after self-cleavage, is shown below.

B. Scheme of the purification process. The bacterial extract containing the fusion protein is loaded onto a chitin-linked (closed ellipse) beads column, where the fusion protein is retained through its chitin binding domain. The column is then incubated with thiols, which results in a specific self-cleavage of the intein which releases the Flo polypeptide. The remainder of the fusion protein is then eluted in detergent buffer to recycle the column.

FIG. 2. Flo Protein Expression

A. SDS PAGE analysis of bacterial extracts. Equivalent fractions of the purification intermediates were loaded as follows: crude extract from IPTG induced cells (lane 1), chitin column flow through (lane 2), eluate of remaining part of the fusion protein, after self-cleavage (intein with chitin binding domain, lane 3), protein molecular weight marker (lane 4). At the left, the upper arrow indicates the fusion protein (61.5 kDa) and the lower arrow indicates the fusion protein after cleavage and elution of Flo (55 kDa).

B. Tris-tricine PAGE analysis of Flo eluate fractions. Lane 1 and 2: 1 and 2 μg of chemically synthesized Flo were loaded, respectively; lane 3 to 8: sequential fractions of Flo elution. At the right, the position of MW markers is as indicated in kDa. The arrow indicates the position of the Flo polypeptide. Trace amount of a polypeptide whose migration corresponds to a dimer of Flo was occasionally noted in highly concentrated fractions (lanes 2 to 6).

FIG. 3. Assay for the Coagulation Activity of Flo.

The glass bead suspension sedimentation assay was performed in a spectrophotometer cells described in the Materials and methods. After 5 minutes stirring, PHYTOFLOC (panel B), synthetic Flo (panel C), or bacterially expressed and purified Flo (panel D), respectively, were added to a final concentration of 20 μg/ml, as indicated by the arrow. In panel A, a similar amount of buffer only was added. Optical density measurement at 500 nm were performed at 1 second intervals. After 15 min, the stirring was stopped. The slopes of the sedimentation curves before and after addition of the compound to be tested, where estimated by linear regression calculations as described in the Materials and Methods, and are shown as straight lines.

FIG. 4. Effect of Flo on E.coli Culture Growth

A. Effect of the PHYTOFLOC seed extract and of bacterial Flo protein. An exponential phase E.coli culture was centrifuged and incubated for 2 hrs at 37° C. in phosphate buffer alone (♦), or in phosphate buffer supplemented by the PHYTOFLOC extract (σ) or by synthetic Flo (ν) at a final concentration of 2 mg/ml. Bacteria were then diluted to A₆₀₀=0.1 in LB growth medium and incubated at 37° C. under agitation. Optical density measurements were then recorded as indicated at 600 nm.

B. Exponentially growing E.coli culture was processed as indicated in (A) except that bacteria were incubated with different concentrations of synthetic Flo in mg/ml: 0 (ν), 0.1 (σ), 0.25 (O), 0.5 (Σ), 1 (λ) or 2 (B). BSA at 2 mg/ml was used as a non specific protein control (♦).

C. Assay for the possible acquisition of a resistance to the antibiotic effect of Flo. E.coli culture consisted either of a fresh culture of bacteria (untreated bacteria) or of a culture previously incubated in presence of the peptide, in two successive rounds, as in FIG. A, where the bacteria that grew eventually were collected (treated bacteria). Untreated cells were then incubated either with buffer (0 mg/ml Flo, ♦) or with Flo (2 mg/ml, λ). Treated cells were incubated for a third cycle in parallel with either buffer (σ) or with 2 mg/ml Flo (ν).

D. Protein synthesis by bacteria incubated or not with synthetic Flo.

Similar volumes of the culture of treated cells shown in Panel C, either incubated with buffer (minus signs) or with 2 mg/ml of Flo (plus signs), were collected at the indicated time, bacteria were precipitated and total cell proteins were separated by SDS-PAGE and stained with coomassie blue.

1. A protein obtained according to a recombinant process which comprises the following steps:
use of a known Moringa protein sequence,
reconstruction of a synthetic gene optimal for expression in E.coli,
design of an expression vector in order to express the protein, preferably as a fusion with an heterologous polypeptide consisting of an intein sequence and a chitin binding domain,
inducing expression with IPTG,
loading of the preparation onto a column, preferably onto a chitin beads-containing column,
elution and recovery of native bacterially expressed proteins, preferably after cleaving of the fusion protein by incubation with thiol-containing reduced compounds.

2. A protein family obtained according to the process disclosed in E proteins—Example 1.

3. A protein family obtained according to the process disclosed in E proteins—Example 2.

4. A protein family obtained according to the process disclosed in E proteins—Example 3.

5. A protein family obtained according to the process disclosed in E proteins—Example 4.

6. A Moringa seed protein family wherein one of its terminal ends starts with the sequence RGPAFRR.

7. Protein partially defined by the alpha-helix Hi as disclosed in FIG. 10.

8. Protein partially defined by the alpha-helix H2 or by the alpha-helix H1 and the alpha-helix H2 as disclosed in FIG. 10.

9. Protein partially defined by the alpha-helix H3 or by H1 and H3 or by H2 and H3 or by H1 and H2 and H3 as disclosed in FIG. 10.

10. A protein family according to any of claim 2 to 5 wherein one of its terminal ends starts with the sequence RGPAFRR.

11. A protein family according to any of the previous claims wherein the protein is chemically synthesized.

12. Process for obtaining a protein family comprising the following steps:
use of a known Modinga protein sequence,
reconstruction of a synthetic gene optimal for expression in E.coli,
design of an expression vector in order to express the protein, preferably as a fusion with an heterologous polypeptide consisting of an intein sequence and a chitin binding domain,
inducing expression with IPTG,
loading of the preparation onto a column, preferably onto a chitin beads-containing column,
elution and recovery of native bacterially expressed proteins, preferably after cleaving of the fusion protein by incubation with thiol-containing reduced compounds.

13. Process for obtaining a protein family as disclosed in E proteins—Example 1.

14. Process for obtaining a protein or protein family as disclosed in E proteins—Example 2.

15. Process for obtaining a protein or protein family as disclosed in E proteins—Example 3.

16. Process for obtaining a protein or protein family as disclosed in E proteins—Example 4.

17. Use of a protein or protein family according any of claim 1 to 11 as a coagulation agent in a fluid.

18. Use of a protein or protein family according to claim 17 wherein the fluid is water.

19. Use of a protein or protein family according to any of previous claim 1 to 11 or 17 or 18 for the manufacture of an antibiotic agent.

20. Use of a recombinant or synthetic protein or protein family from Moringa Oleifera for the manufacture of an antibiotic agent.

21. Use of a natural protein or extract from Moringa Oleifera for the manufacture of an antibiotic agent.

22. Oral use of the protein or protein family according to claim 20 or 21.

23. Topical use of the protein or protein family according to claim 20 or 21.
